(19) United States
(12) Patent Application Publication
(10) Pub. No.: US 2006/0223779 A1
(43) Pub. Date: Oct. 5, 2006

(54) Method of screening for inhibitors of human Fatty Acid-CoA Ligase 4

(76) Inventors: Stephen M. Prescott, Salt Lake City, UT (US); Yang Cao, Salt Lake City, UT (US)

Correspondence Address: MADSON & AUSTIN;GATEWAY TOWER WEST, SUITE 900, 15 WEST SOUTH TEMPLE, SALT LAKE CITY, UT, 84101, US

(21) Appl. No.: 11/445,723

(22) Filed: Jun. 2, 2006

Related U.S. Application Data
(62) Division of application No. 10/362,222, filed on Feb. 20, 2003, now abandoned.

(51) Int. Cl. A61K 48/00; A61K 31/192; G01N 33/574; C12Q 1/68

(52) U.S. Cl. 514/44; 435/6; 435/7.23; 514/569; 514/575

(57) ABSTRACT

The present invention provides a method of screening for potential cancer chemotherapeutic and chemopreventive agents which act by modulating the activity of the Fatty Acid-CoA Ligase 4 (FACL4) gene product in humans. This invention also provides methods of administering such agents to treat cancer by inhibiting the activity of the FACL4 gene product, thus raising the cellular level of free arachidonic acid and triggering arachidonic acid-mediated apoptosis. The present invention further provides a method for diagnosing cancers, including colon cancer.

### CROSS-REFERENCE TO RELATED APPLICATION

This application is a divisional of U.S. Application Ser. No. 10/362,222, filed Feb. 20, 2003, which was the National Stage of International Application No. PCT/US00/22920, filed Aug. 21, 2000, which applications are incorporated by reference.

### BACKGROUND OF THE INVENTION

The present invention relates to chemotherapeutic treatments for cancer. More specifically, the invention relates to screening assays for cancer chemotherapeutic agents which modulate the activity of the enzyme Fatty Acid Co-A Ligase 4 (“FACL4”) in humans, to novel treatments for colon cancer which involve the administration of agents discovered using said assays, and to new methods for diagnosing colon cancer by detecting the levels of FACL4 activity in tissue samples taken from a patient being screened for colon cancer.

Cancer is a disease in which normal body cells are changed, becoming able to multiply without regard to normal cellular restraints and to invade and colonize areas of the body normally occupied by other cells. See B. Alberts et al., Molecular Biology of the Cell 1255-1294 (3d ed. 1994). According to the American Cancer Society, one-half of all American men and one-third of all American women will at some point in their lives develop cancer.

Due to the ability of cancer cells to spread and rapidly proliferate, it is difficult to treat cancer patients by attempting to selectively kill cancerous cells. Some have compared the difficulty of this task to the difficulty of completely ridding a garden of weeds. As with weeds, if only a few cancer cells are left untouched by treatment, they may again spread throughout the body, causing a recurrence of the disease. See id. at 1267. Current treatments for cancer include surgery and therapies using chemicals and radiation. The effectiveness of these treatments is often limited, however, since cancer cells that have spread from the original tumor site may be missed by surgery and radiation, and since chemical treatments which kill or disable cancer cells are often capable of causing similar damage to normal cells. See id.

Hope for better treatments for cancer focuses on obtaining a better understanding of carcinogenesis—the series of events which transforms a normal cell into a cancer cell. It is hoped that such an understanding will help researchers and physicians direct treatments solely toward cancer cells or their precursors, thus preventing or treating cancer and avoiding damage to healthy body tissues.

One way researchers are learning about carcinogenesis is by evaluating how chemical agents which are known to be useful in preventing or treating cancer act to prevent cancer in the body. One result of such research is the discovery that some cancer-preventing (“chemopreventive”) and cancer therapeutic (“chemotherapeutic”) agents act on cellular enzymes which metabolize arachidonic acid in cells. Recent studies have shown that arachidonic acid metabolism plays an important role in the development of cancer. See M. Tsujii & R. N. DuBois, Cell, 83:493-501 (1995); H. Sheng et al., 99 J. Clin. Invest. 2254-2259 (1997); and S. M. Prescott & R. L. White, 87 Cell 783-786 (1996). Arachidonic acid (“AA”; 20:4, n-6) is an essential polyunsaturated fatty acid which is oxidized in cells to form eicosanoids including prostaglandins and leukotrienes.

One cellular enzyme which metabolizes AA is named COX-2. COX-2 is an enzyme involved in the synthesis of prostaglandins from AA. It has been shown to be specifically involved in colon carcinogenesis. Like other enzymes, COX-2 can be successfully chemically inhibited. Indeed, it has been shown in recent population studies that regular use of nonsteroidal anti-inflammatory drugs (“NSAIDs”), which inhibit COX-2, lowers colon cancer risk. See M. J. Thun et al., 325 New Engl. J. Med. 1593-1596 (1991). Use of such NSAIDs also helps to prevent carcinogen-induced cancer in animals. See F. M. Giardiello et al., 328 New England J. Med. 1313-1316 (1993); and C. S. Williams et al., 18 Oncogene 7908-7916 (1999). Moreover, COX-2 has been shown to be upregulated in colon cancer, as has the reticulocyte type 15-lipoxygenase (“LOX”), another enzyme which metabolizes AA. This upregulation of several AA-metabolizing enzymes in cancer cells suggests that multiple pathways of AA metabolism are activated during carcinogenesis.

Several lines of evidence indicate that the level of free AA in cells can regulate apoptosis, the cellular mechanism for programmed cell death. One such line of evidence is found in a mutant cell line shown to be resistant to cell death from exposure to tumor necrosis factor alpha (“TNFα”). TNFα is a protein shown to preferentially kill cancer cells. In this mutant cell line, the activity of cytosolic phospholipase A2 (“cPLA₂”) was significantly reduced, thereby also reducing levels of AA synthesis. This suggests that blocking endogenous AA release in cells, and thus keeping the levels of free AA in cells low, renders cells resistant to TNFα killing. See M. Hayakawa et al., 268 J. Biol. Chem. 11290-11295 (1993).

Additional evidence is provided by another line of TNFα killing-resistant cells in which a defect in the rate-limiting enzyme for AA biosynthesis, Δ6-desaturase, has been reported. See T. Reid et al., 266 J. Biol. Chem. 16580-16586 (1991). This defect would similarlykeep AA levels in cells low, thus preventing TNFα killing.

Without being bound to any particular theory, one interpretation of the above results is that a reduced cellular AA pool sets a limitation on the level of endogenous AA that can accumulate in response to exposure to TNFα. This limitation on the amount of free cellular AA appears to confer resistance to cell death. This interpretation does not distinguish between an effect of the cellular levels of free AA itself or that of a downstream product like an eicosanoid, however.

This interpretation seems to be further supported by a study involving lipoxygenases. This study reported that the administration of inhibitors of 12-LOX, a lipoxygenase involved in converting arachidonic acid into a leukotriene, induced apoptosis in rat carcinosarcoma cells. See D. G. Tang et al., 93 Proc. Natl. Acad. Sci. 5241-5246 (1996). Likewise, the administration of NSAIDs has been shown to yield a similar response in colon cancer cell lines. Indeed, apoptosis may be induced by simply adding exogenous AA to the cells. See T. A. Chan et al., 95 Proc. Natl. Acad. Sci. 681-686 (1998).

The mechanism by which these inhibitors induce cell death is not completely understood, although research has shown that blocking arachidonate-phospholipid remodeling by inhibitors of CoA-independent transacylase (CoA-IT) induced apoptosis. This corresponded to an accumulation of cellular free AA like that which resulted from the inhibition of COX-2 and LOX. See M. E. Surette et al., 35 Biochemistry 9187-9196 (1996); and M. E. Surette et al., 20 Carcinogenesis 757-763 (1999). Successful inhibition of AA metabolism appears to facilitate the accumulation of AA in cells, thus facilitating the induction of tumor cell death by apoptosis.

This research has spawned much investigation into methods of inhibiting enzymes which metabolize AA. Inhibitors are known for COX-2 and other enzymes which metabolize AA. Other studies of enzymes which metabolize AA in humans have centered on the enzyme human Fatty Acid CoA Ligase 4 (“FACL4”). FACL4 is upregulated in colon adenocarcinoma cells. Moreover, as with other AA-metabolizing enzymes, inhibiting FACL4 induces apoptosis in colon adenocarcinoma cells. These properties make FACL4 inhibition a promising avenue of research for and application of prospective cancer treatments and give new hope to cancer sufferers. However, little is known about agents which inhibit FACL4, and there are no known treatments for cancer to date which utilize this pathway. No use has been made of the elevation of FACL4 expression in cancer cells as a diagnostic method.

From the foregoing, it will be appreciated that it would be a significant advancement in the art to provide additional effective methods for regulating AA-mediated apoptosis in cancer cells. Specifically, it would be an advancement in the art to provide methods for screening for chemotherapeutic or chemopreventive agents which act by modulating the activity of FACL4 in cells, including cancer cells. It would be a further advancement in the art to provide novel treatments for colon cancer involving the inhibition of FACL4 by the administration of chemotherapeutic or chemopreventive agents discovered using such assays. Similarly, it would be an advancement in the art to provide new methods for diagnosing colon cancer by detecting the levels of FACL4 activity in tissue samples. Such methods are disclosed herein.

### BRIEF SUMMARY OF THE INVENTION

The present invention relates to chemopreventive and chemotherapeutic treatments for cancer. More specifically, the present invention relates to methods of screening compounds for cancer chemotherapeutic activity. Said methods of screening detect agents which modulate the activity of the enzyme Fatty Acid Co-A Ligase 4.

In certain embodiments, said methods comprise contacting cells which express or overexpress the FACL4 gene product with the compound to be tested, and then measuring the level of inhibition of the FACL4 gene product in the cells. The inhibition of the gene product is measured in any of the numerous ways known in the art, including, but not limited to monitoring the rate and amount of radiolabeled AA esterified by FACL4 in compound-treated and untreated cells or comparing rates of apoptosis among compound-treated cells with those of untreated cells. See D. B. Wilson et al., 257 J. Biol. Chem. 3510-3515 (1982); and Y. Cao et al., 49 Genomics 327-330 (1998). In certain other embodiments, said methods comprise contacting a genetically engineered cell in which the expression of FACL4 is suppressed with a test compound and measuring inhibition of the function of the FACL4 gene product in the cell. One could also test for FACL4 inhibitors using mouse models, for example in mice bearing xenografted tumors, in transgenic mice or cells derived therefrom that overexpress FACL4, or in FACL4 knock-out mice. In embodiments of the present invention, test compounds which inhibit the function of the FACL4 gene product are potential cancer chemotherapeutic agents.

The invention also relates to providing novel treatments for colon cancer by administering chemotherapeutic or chemopreventive agents discovered using the assay methods disclosed above to a human in need of such treatment. Thus, said novel treatments for colon cancer comprise inhibiting FACL4 in a human by administering a FACL4 inhibitor. In specific embodiments, these treatments comprise administering triacsin C to a human. As used herein, “effective amount” means an amount of a drug or pharmacologically active agent that is nontoxic but sufficient to provide the desired local or systemic effect and performance at a reasonable benefit/risk ratio attending any medical treatment. In other embodiments, said treatments comprise administering a FACL4-inhibiting compound to a human, wherein the activity of the compound does not result in significant toxic effects to the human. In yet another embodiment, said treatments comprise administering a compound which inhibits FACL4 to a human in need of such treatment, wherein the ability of the compound to inhibit FACL4 is determined by contacting it with a cell genetically engineered to express or overexpress a FACL4 gene product and determining whether the compound inhibits the FACL4 gene product.

Further, the invention relates to new methods for diagnosing colon cancer by detecting the levels of FACL4 activity in tissue samples. In some embodiments, said methods comprise isolating a sample of tissue suspected to be cancerous, evaluating levels of FACL4 expression in the cells of the tissue, and comparing the levels of FACL4 expression with those of a control cell. Evaluation of the levels of expression of FACL4 may be conducted through quantitative RT-PCR, immunostaining, in situ hybridization, immunofluorescence, Western blotting, or other methods commonly known in the art. Further, the control cell used in these methods may be selected from non-cancerous colon cell lines known in the art, or may simply be a cell from a tissue sample thought to be normal taken from the same individual.

These and other features and advantages of the present invention will become more fully apparent from the following detailed description.

### BRIEF SUMMARY OF THE DRAWINGS

FIG. 1: The expression of FACL4 and COX-2 is increased in colon adenocarcinomas and cell lines. (A) Expression in adenocarcinomas. Quantitative RT-PCR was performed on RNAs isolated from 24 pairs of colon adenocarcinomas and adjacent normal tissues. A representative RT-PCR quantitation from two patients is shown. The top panel illustrates amplification with the FACL4 amplicons and the bottom with the COX-2 amplicons. FACL4 was amplified for 32 and 36 cycles, and COX-2 was amplified for 34 and 38 cycles. T denotes colon adenocarcinoma tissue, while N denotes normal colon tissue from the same patient. (B) Expression in colon cancer cell lines. The legends on top of the gel indicate the cell line from which RNA was extracted.

FIG. 2: Triacsin C sensitizes arachidonic acid-induced apoptosis (A) and synergizes with NSAIDs in inducing apoptosis (B). HT 29 Cells were plated in 96 wells. Triacsin C and AA were added (in A), or triacsin C with either indomethacin (Indo) or sulindac (Sul) (in B) were added at the indicated concentrations. Indomethacin and sulindac are both cyclooxygenase (COX) inhibitors. Apoptosis was determined by Cell Death ELISA 44 hr later (A), and 72 hr later (B), respectively. Control cells, (hereinafter “Ctl”) are considered to be cells which did not receive NSAIDs treatment. The values shown are the means of triplicate determinations.

FIG. 3: Arachidonic acid induces apoptosis via activation of caspase 3. (A) Determination of cell survival. AA was added to 293 cells at the indicated concentrations and the surviving fraction of cells was determined after 44 hr. The values shown are relative to the viability of cells that were not exposed to AA. (B) Increased apoptosis in cells exposed to AA. Cells were treated with AA (300 μM) or palmitic acid (300 μM) under the same conditions as in A, and then apoptosis was detected with an ELISA. The mean values from triplicate determinations are shown. In multiple experiments the concentration of AA that induced apoptosis in a substantial fraction of the cells varied between 200 and 300 μM (C) In situ detection of apoptosis. The 293 cells were treated with AA (300 μM; +AA) or vehicle alone (−AA) for 44 hr. The nuclei were stained with DAPI, and DNA strand breaks in individual cells were detected with a fluorescent TUNEL assay. The upper panels are the same field of cells (−AA) photographed under UV (left panels) and FITC filters (right panels). The lower panels illustrate apoptotic cells (+AA). The magnification is 80×. (D) Immunostaining with an anti-active caspase 3 antibody. Upper panels, control cells (−AA); lower panels, cells exposed to 300 μM AA. Left panels, DAPI staining; right panels, immunostaining with an antibody selective for active caspase 3. The magnification is 100×. (E) Blocking AA-induced apoptosis by inhibition of caspase 3. The 293 cells were preincubated with caspase inhibitors (including DMQD, a caspase 3 inhibitor, and YVAD, a caspase 1 and 4 inhibitor (Peptide Institute)) for 1 hr at the indicated concentrations, AA (300 μM) was added, and apoptosis was determined 44 hr later. The values represent the means from triplicate determinations.

FIG. 4: Over-expression of COX-2 or FACL4 inhibits apoptosis. (A) Conditional expression level of FACL4 and COX-2 in stably transfected lines. Cells were treated with (+) or without (−) ponasterone (1 μg/ml) for 48 hr, and Western blotting was carried out with anti-FACL4 and anti-COX-2 antibodies, respectively. The loading was normalized with an anti-β actin antibody (ICN Biomedicals). (B) Reduction of AA-induced apoptosis by over-expression of COX-2, FACL4, and both together. The 293 cells stably transfected with an empty control vector (Ctl), FACL4 cDNA, COX-2 cDNA, and both FACL4 and COX-2 cDNAs were uninduced (−Pon) or induced for over-expression (+Pon) as in A. AA (300 μM) was added after 24 hr of induction and apoptosis was determined 44 hr later. The increase in apoptosis induced by AA varied between 6- and 20-fold relative to the signal in cells without treatment. The plot is a comparison of the relative apoptosis levels among the stable lines and is representative of three independent experiments. The values shown are the averages of triplicate determinations. (C) Requirement for the catalytic activity for the COX-2 mediated prevention of apoptosis. Cells were uninduced (−Pon) or induced for over-expression as above. The mutant COX-2 was expressed at a similar level to the wild-type, as assessed by immunoblotting, but there was no increase above background in the amount of prostaglandin synthesized. COX inhibitors were administered 6 hr later as indicated. AA (300 μM) was then added after 2 hr and apoptosis was determined 44 hr later. The plot is prepared as in B, and is a representative result from three independent experiments, in which triplicates were performed. Wt=wild-type, Mut=mutant, Met=50 μM 6-methoxy-2-naphthyl acetic acid, Indo=10 μM indomethacin. (D) Reduction of prostaglandin synthesis by co-expression of FACL4 with COX-2. The COX-2, FACL4/COX-2 stable and control cells carrying an empty vector were induced for 48 hr, washed and incubated with 20 μM AA at 37° C. for 30 min. The amount of PGE₂ synthesized was determined by ELISA. Four additional clones of the FACL4/COX-2 double stable cells were examined, and all secreted a lower level of PGE₂—ranging from 16% to 56%—than the COX-2 single stable lines. (E) The levels of free AA in the stable lines. The [³H] AA released to the supernatant was determined and the values shown are averages from multiple experiments (n≧4, P<0.05).

FIG. 5: Enzymatic “sinks” for free arachidonic acid protect against TNFα_ and calcium ionophore_mediated cell killing. (A) Determination of cell survival. The cells were induced (+Pon) or not (−Pon) for 24 hr, and TNFα (1 ng/ml) or the calcium ionophore A23187 (5 μM) was added, and the cells were incubated for another 72 hr. The surviving fraction was determined and is plotted relative to that of the same line without TNFα or A23187 treatment. Ctl denotes control cells harboring an empty vector, while FACL4/COX-2 denotes the double stable line. (B) Measurement of the free AA. The FACL4/COX-2 cells were prelabeled with [³H] AA, induced, and stimulated with A23187 (5 μM), and [³H] AA release was measured at the indicated time intervals. The values are from one experiment that is representative of four independent determinations.

FIG. 6: Cells exposed to AA show increased lipid peroxidation; apoptosis is blocked by antioxidants and Bcl-2. (A) The level of lipid oxidation products. The 293 cells were treated with AA at the indicated concentrations for 48 hr. The data presented are from one experiment that is representative of two independent experiments. (B) Attenuation of AA-induced apoptosis by antioxidants. Antioxidants were applied to 293 cells at the indicated concentration for 1 hr, and then AA (200 μM) was added. Apoptosis was determined after 44 hr and is shown relative to the level in control cells. (C) Inhibition of AA-induced apoptosis by Bcl-2. Cells were transfected with the empty vector (Ctl) or with a cDNA encoding Bcl-2 and then were exposed to AA (300 μM). The level of apoptosis was determined after 44 hr. The values shown indicate the apoptosis level relative to control cells, and represent the means of triplicate determinations (P<0.05). The expression of Bcl-2 was detected by Western blotting. (D) The level of Bcl-2 expression in the stable cells. The stably transfected cells were induced, or not, with ponasterone for 24 or 48 hr. Cell lysates were blotted with an antibody to Bcl-2. Normalization was performed with an anti-β-actin antibody.

These drawings only provide information concerning typical embodiments of the invention and are not therefore to be considered limiting of its scope.

### DETAILED DESCRIPTION OF THE INVENTION

The present invention provides screening assays for cancer chemotherapeutic agents which modulate the activity of the enzyme Fatty Acid Co-A Ligase 4. The invention also provides novel treatments for colon cancer which involve the inhibition of FACL4 by the administration of chemotherapeutic or chemopreventive agents discovered using such assays. Further, the invention provides new methods for diagnosing colon cancer by detecting the levels of FACL4 activity in tissue samples.

All publications, patents, and patent applications cited herein are hereby incorporated by reference.

### Definitions

By “vector” is meant any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., 52 Virology 456 (1973); Sambrook et al., Molecular Cloning: A Laboratory Manual, Cold Spring Harbor Laboratories, New York (1989); Davis et al., Basic Methods in Molecular Biology, Elsevier, (1986); Chu et al., 13 Gene 197 (1981); and J. D. Watson et al., Recombinant DNA, 213-234 (1996). Such techniques can be used to introduce one or more exogenous DNA moieties, such as a nucleotide integration vector and other nucleic acid molecules into suitable host cells. The term captures chemical, electrical, and viral-mediated transfection procedures, including, as examples, calcium phosphate co-precipitation (Graham et al., 52 Virology 456-467 (1973)), direct microinjection into cultured cells (M. R. Capecci, 22 Cell 479-488 (1980)), electroporation (Shigekawa et al., 6 BioTechniques 742-751 (1988)), liposome-mediated gene transfer (Mannino et al., 6 BioTechniques 682-690 (1988)), lipid-mediated transfection (Felgner et al., 84 Proc. Natl. Acad. Sci. 7413-7417 (1987)), and nucleic acid delivery using high-velocity microprojectiles (Klein et al., 327 Nature 70-73 (1987)).

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “RT-PCR” denotes the reverse transcriptase polymerase chain reaction used and known in the art. As is known in the art, RT-PCR is a variation on a basic method of amplifying a target DNA molecule by using a polymerase and primers which have nucleotide sequences complementary to the sequence of the DNA flanking the desired target region of the DNA. Successive cycles of the process can be used to increase the amount of DNA present since the strands produced in a first cycle can be used as templates for production in later cycles. RT-PCR combines this basic method with the reverse transcription of mRNA to form cDNA, which may then be amplified by the procedure just described. See R. C. King, A Dictionary of Genetics, New York, Oxford University Press 268 (1997).

The term “amplicon” is used to mean a segment of genetic material which forms many linear copies upon exposure to a compound which inhibits the function of a gene found in the segment.

The term “over-expression” as used herein denotes that a given gene product is being expressed in a cell or set of cells that have been engineered to express the gene product at a rate higher than in a comparable cell or set of cells that have not been so engineered. The rates of overexpression in said cells vary from the original levels by 2-fold, 5-fold, and 10-fold, with 10-fold being preferred.

### General Methods

The invention stems from the discovery that fatty acid CoA ligase 4 (FACL4), like COX-2 and LOX, is upregulated in colon adenocarcinoma. See Y. Cao et al., 49 Genomics 327-330 (1998). This upregulation of these enzymes suggests that colon cancer causes alterations to existing AA metabolic pathways which cumulatively lower the level of free AA in a cell, thereby preventing apoptosis. This hypothesis was tested by establishing human epithelial lines with stable, inducible expression of FACL4 and/or COX-2. Using these cell lines, it was determined that AA is a signal for apoptosis. These assays further showed that the induction of apoptosis by NSAIDs and other inhibitors of AA metabolism is a consequence of the accumulation of AA due to the inhibition of AA-metabolizing agents. These assays further showed that over-expression of COX-2 and FACL4, like that which occurs in colon and perhaps other cancers creates a “sink” for AA, thereby removing a pro-apoptotic signal and promoting carcinogenesis. Pharmacological manipulation of the cellular level of free AA to induce apoptosis could thus be a general approach to killing transformed cells. This could be achieved through the inhibition of enzymes which metabolize AA, such as COX-2 and FACL4.

In order to discover compounds which block the metabolism of AA by FACL4, human epithelial cell lines were developed with stable, inducible expression of FACL4, COX-2, and both FACL4 and COX-2. These cell lines were used in methods of screening for compounds which inhibit FACL4. Such methods comprise contacting a test composition with a cell expressing or overexpressing the FACL4 gene product and measuring the inhibition of the FACL4 gene product by the compound relative to that of a non-contacted cell. Many methods may be used to measure the inhibition of the FACL4 gene product by the test compound, including tracking the metabolism of radiolabeled AA in FACL4-treated and non-FACL4-treated cells. Similarly, pulse-chase methods such as that used in Example 5 can show relative amounts of AA metabolism occurring in a set of cells versus that of a control. Further, methods stemming from the apoptosis-inducing effects of AA pooling such as comparing rates of apoptosis in FACL4-treated cells with those of non-FACL4-treated cells and comparing apoptosis rates of FACL4-treated and non-treated cells after exposure to exogenous AA.

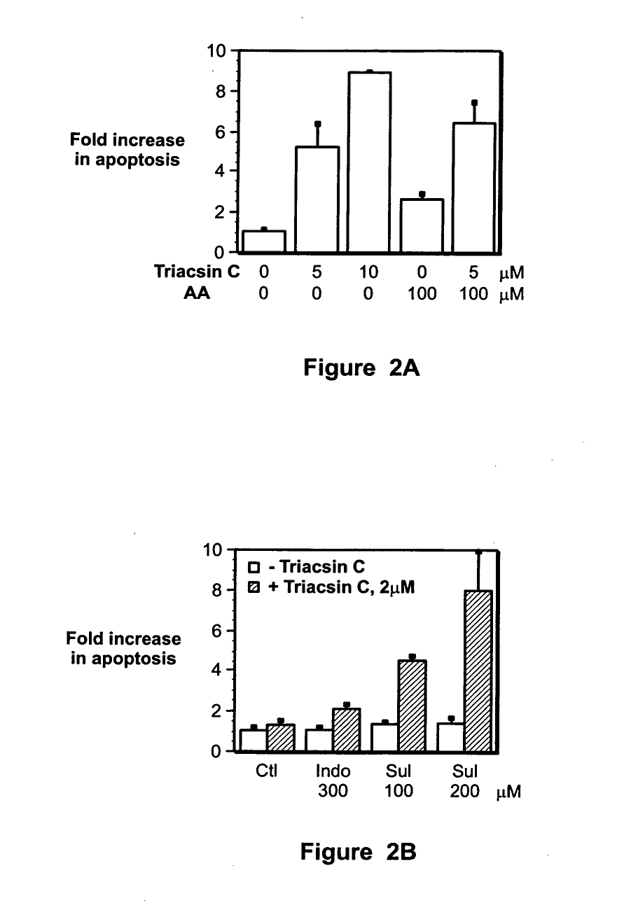

Further, the compounds discovered using the above assays may be used to treat colon cancer in a human. As seen, for example, in Example 2, known inhibitors of FACL4 such as triacsin C may be used to sensitize cells to AA-induced apoptosis. Indeed, there is a synergistic effect in destroying cancer cells when both FACL4 and COX-2 are inhibited, as seen in FIG. 2b.

Also, as seen in Example 1, colon adenocarcinomas express FACL4 at much higher levels than normal cells. This suggests that evaluating the levels of FACL4 expression in a sample of suspect tissue and comparing it to the levels found in a sample of normal tissue, a normal cell line, or other control would serve as a method of diagnosing colon cancer.

The concentrations of AA that induced apoptosis in the in vitro experiments described herein are higher than the steady state levels that have been measured in normal biological fluids, but the concentration in cells is unknown and might approach such levels transiently under certain circumstances in vivo. For example, approximately 5 to 20 nmol of AA are released from 10⁹ platelets in 1 min upon stimulation by thrombin. See E. J. Neufeld & P. W. Majerus, 258 J. Biol. Chem. 2461-2467 (1983).

The induction of apoptosis is not a detergent effect since other fatty acids, such as oleic and palmitic acids, did not cause the same response. Moreover, the AA-initiated apoptosis can be inhibited by removal of AA metabolically as shown in our experiments in which COX-2 and FACL4 were over-expressed.

COX-2-specific inhibitors SC-58125 and NS398 have been reported to enhance the induction of apoptosis in colon and prostate cancer cells by down-regulating the anti-apoptotic protein Bcl-2 (See H. Sheng et al., 58 Cancer Res. 362-366 (1998); and X. H. Liu, et al., 58 Cancer Res. 4245-4249 (1998)), whereas 15-LOX was found to block apoptosis by upregulating Bcl-2. See E. Nishio & Y. Watanabe, 122 Br. J. Pharmacol. 1516-1522 (1997). We have discovered that overexpressing COX-2 and/or FACL4 blocked apoptosis, and was paralleled by an increased expression of Bcl-2. In addition, over-expression of Bcl-2 suppressed the AA-induced apoptosis. These suggest that the expression of Bcl-2 is regulated by the level of free AA, and that the Bcl-2 dependent pathways play a crucial role in AA signaled apoptosis.

It has been debated whether the augmented AA metabolism is an initiator or a consequence of tumor growth. The studies with COX-2 and its inhibitors strongly support that the induction of COX-2 is a critical step in carcinogenesis and tumor progression. The pro-neoplastic effects of activating COX-2 and other AA metabolic pathways are multiple, but one important mechanism is the inhibition of apoptosis. In the case of colon physiology, apoptosis is a normal event to conclude the life cycle of intestinal epithelial cells. The diversion of AA by the induced enzymes in colon cancer lowers the level of free AA, and thereby promotes tumor growth by attenuating apoptosis. The specific eicosanoid products of AA metabolism, like prostaglandins and leucotrienes may also contribute to the transformation through enhancing cell adhesion and proliferation. Nonetheless, the inhibition of apoptosis by coordinated activation of AA metabolic “sinks” is a vital mechanism to promote tumor growth, and our findings implicate that development of specific and non-toxic inhibitors targeted to the AA-metabolizing pathways may provide novel approaches for therapeutic intervention.

### EXAMPLES

The following examples are given to illustrate various embodiments which have been made with the present invention. It is to be understood that the following examples are not comprehensive or exhaustive of the many types of embodiments which can be prepared in accordance with the present invention.

### Materials and Methods

Tumor tissues and Reverse Transcription PCR. Total RNA was prepared from surgically removed colon adenocarcinoma and the adjacent normal tissues from the same patient by Trizol Reagent (Life Technologies, Inc.). Colon cancer cell lines were from ATCC. RT-PCR was carried out using primers corresponding to the coding sequence of human FACL4 or COX-2, and β-actin primer-competimer mix (Ambion). The sizes of the amplified fragments for FACL4 and COX-2 are 441 bp and 480 bp, and the β-actin primers amplify a fragment of 294 bp.

Cell Death Detection ELISA. Cells were plated on 96 well-plates at 1×10⁴ cells per well. AA was applied and apoptotic death was determined after 44 hr with the Cell Death Detection ELISA (Boehringer Mannheim).

Stable cell lines. The ecdysone inducible system (Invitrogen) was used to construct stable lines over-expressing human FACL4, COX-2, COX-2 mutant, and both FACL4 and COX-2. COX-2 mutant cDNA expresses a catalytically inactive COX-2 with change of a single amino acid residue (L547K). The cDNAs were cloned into pIND(SP1) (Neomycin^{r}). The cDNA constructs, or the empty vector pIND(SP1) were transfected into EcR293 cells, which were then selected in 400 μg/ml geneticin and 400 μg/ml zeocin. A number of single colonies were screened for over-expression of FACL4, COX-2 wild type, and COX-2 mutant by immunoblotting against anti-FACL4 (See Y. Cao et al., 467 FEBS Lett. 263-267 (2000)), and anti-COX-2 antibodies (a gift from Dr. Jacques Maclouf), and by measurement of the CoA ligase activity for FACL4 (See D. B. Wilson et al., 257 J. Biol. Chem. 3510-3515 (1992)), or the PGE₂ secretion for COX-2 wild type (ELISA assay, Assay Design, Inc.). The double stable line was generated by stably transfecting the COX-2 stable cells with another pIND(SP1)-FACL4 cDNA construct, in which the FACL4 cDNA was cloned into a pIND(SP1) (hygromycin^{r}) vector. The cells were selected in 400 μg/ml geneticin, 400 μg/ml zeocin, and 50 μg/ml hygromycin B. The positive colonies were screened as described above.

Cell survival. Cell viability was determined with the crystal violet staining (See M. Hayakawa et al., 268 J. Biol. Chem. 11290-11295 (1993).

In situ cell death detection. Cells were plated on 8 well-glass chamber slides precoated with poly-L-lysine. AA (300 μM) was added, and the fluorescent TUNEL assay was performed 44 hr later using the in situ cell death detection kit (Boehringer Mannheim).

Arachidonic acid release. Cells were plated on 6 well-plates precoated with poly-L-lysine at 8×10⁵ cells per well. They were then prelabeled with [³H] AA (0.4 μCi/ml medium) (91.8 Ci/mmol, 100 μCi/ml, NEN) for 24 hr. After a wash, serum-free DMEM supplemented with 1% BSA (fatty acid free) was added. A small portion of the medium was withdrawn at time intervals, and free fatty acids were extracted with the isopropyl alcohol/heptane/2M H₂SO₄ (40:10:1) cocktail (See V. Dole & H. Meinertz, 235 J. Biol. Chem. 2595-2599 (1960). The upper heptane phase was collected and the [³H] labeled AA was determined by a scintillation counter. The medium was extracted 28 hr later and AA release was determined again. The percentage of AA release (+P/−P) was calculated by first subtracting released AA after 4 hr (background) from that at 32 hr in the same dish, secondly dividing the release level after ponasterone treatment (+P) with that of control (−P) in the same stable line. The AA release stimulated by A23187 was calculated by subtracting the released AA by unstimulated cells, from that by A23187-stimulated counterparts.

Lipid peroxidation assay. Cells were plated and treated with AA for 48 hr. They were then suspended in 300 μl H₂O and the cell lysates were prepared by repeatedly freezing and thawing. The cellular malondialdehyde (MDA) and 4-hydroxynonenal (4-HNE) levels were determined with the lipid peroxidation assay kit (Calbiochem).

Transfection with Bcl-2 cDNA construct. The cells were transfected with human Bcl-2 cDNA construct using LipofectAMINE (Life Technologies, Inc.). Two μg of Bcl-2 cDNA and 1 μg of luciferase cDNA constructs were transfected into ˜4×10⁵ cells. The cells were harvested 48 hr later and replated at 1×10⁴ cells per well on 96-well plates. The transfectants were exposed to 300 μM AA, and apoptosis was examined 44 hr later. A portion of the transfectants was lysed, and the luciferase activity was determined to monitor the transfection efficiency. The expression level of Bcl-2 was examined by immunoblotting (antibody from Boehringer Mannheim).

Indirect fluorescent immunostaining. Cells were cultured in poly-L-lysine precoated 8-chamber slides, and were fixed with 3.7% paraformaldehyde. The slides were then blocked and the anti-active caspase 3 antibody (1:200 dilution, Pharmingen) was applied, followed by incubation with FITC-conjugated goat anti-rabbit secondary antibody (1:250 dilution, Jackson ImmunoResearch).

### Example 1

### The Expression of FACL4 Is Increased in Colon Adenocarcinomas

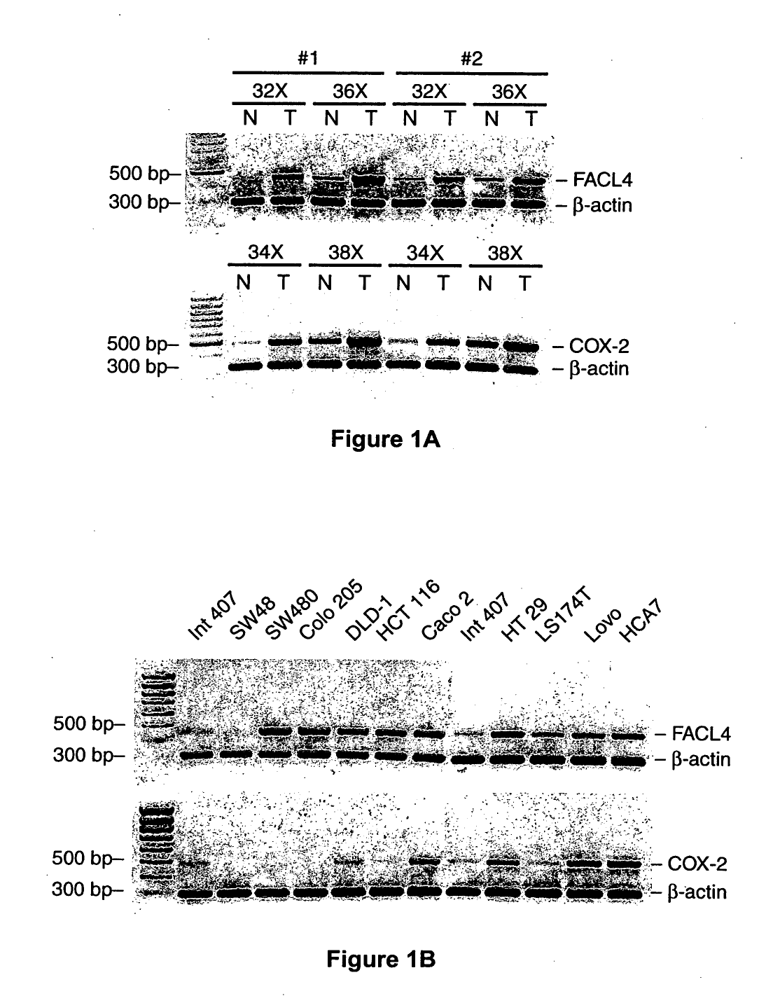

We previously cloned the human isoform 4 of fatty acid CoA-ligase that encodes an enzyme that highly prefers AA and that converts it into arachidonoyl coenzyme A ester. (See Y. Cao et al., 49 Genomics 327-330 (1998)). Since FACL4 and COX-2 both use AA as substrate, and since COX-2 was shown to be induced in colon cancer (See C. E. Eberhart et al., 107 Gastroenterology 1183-1188 (1994)), we asked if FACL4 is also upregulated in colon adenocarcinomas. By quantitative RT-PCR using primers specific for COX-2 and FACL4, we found that in addition to COX-2, the expression of FACL4 was significantly increased in the colon adenocarcinoma compared to the adjacent normal tissue from the same patient. We observed a 2- to 14-fold increase in the expression of FACL4 in 23 of 24 colon adenocarcinoma samples, and a representative result from two patients is shown in FIG. 1A. We also found that many colon carcinoma cell lines express a significantly higher level of FACL4 than in the intestinal epithelial line Int 407 (FIG. 1B).

### Example 2

### A FACL Inhibitor Sensitizes a Cell to AA-Induced Apoptosis and Synergizes with NSAIDs in Inducing Apoptosis

Previous reports indicated that inhibition of COX-2 by NSAIDs in transformed cells results in apoptosis. We evaluated whether triacsin C, an inhibitor of FACL, has a similar effect. We found that triacsin C induced apoptosis in a concentration-dependent manner in HT 29 cells; at 10 μM it caused a 9-fold increase compared to control cells (FIG. 2A). Furthermore, exogenous AA induced apoptosis at concentrations from 100 to 300 μM, and triacsin C sensitized cells to AA-induced apoptosis (FIG. 2A). This suggests that the induction of apoptosis by triacsin C is mediated through inactivation of the FACL4 pathway and subsequently, an accumulation of free AA in cells. We further tested whether blocking these two metabolic pathways simultaneously exerts a synergistic effect on apoptosis. We found that triacsin C at a concentration (2 μM) that otherwise did not cause cell death in HT 29 cells, induced apoptosis when indomethacin, a COX inhibitor, was added. The apoptosis-inducing effect of triacsin C was increased by 4-8 fold in the presence of sulindac, another COX inhibitor (FIG. 2B). A similar synergy was detected in human kidney epithelial 293 cells (not shown). Thus, it is likely that the signal for apoptosis is an elevated level of AA, and triacsin C and NSAIDs promote apoptosis by blocking the metabolic removal of free AAs.

### Example 3

### Arachidonic Acid Induces Apoptosis in Epithelial Cells and Involves Activation of Caspase-3

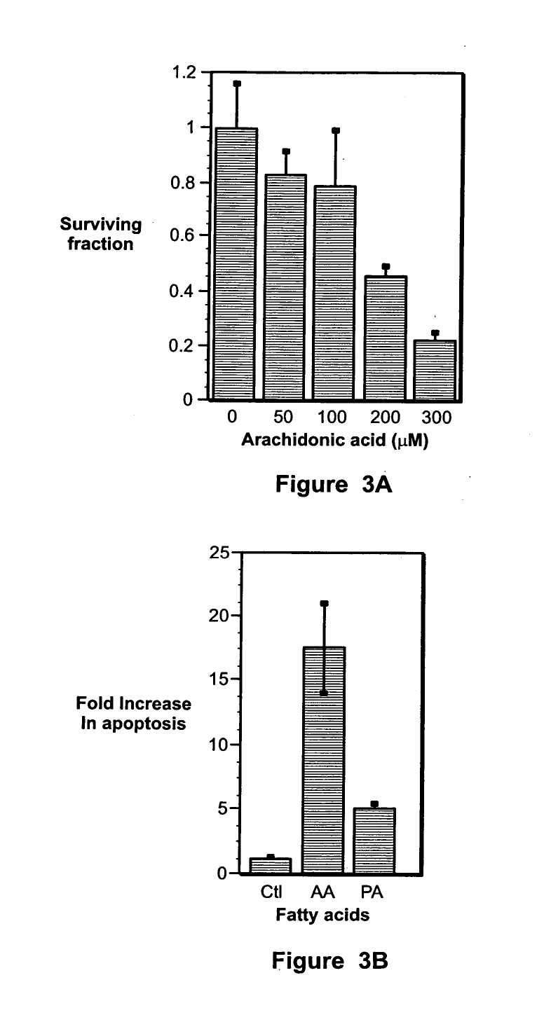

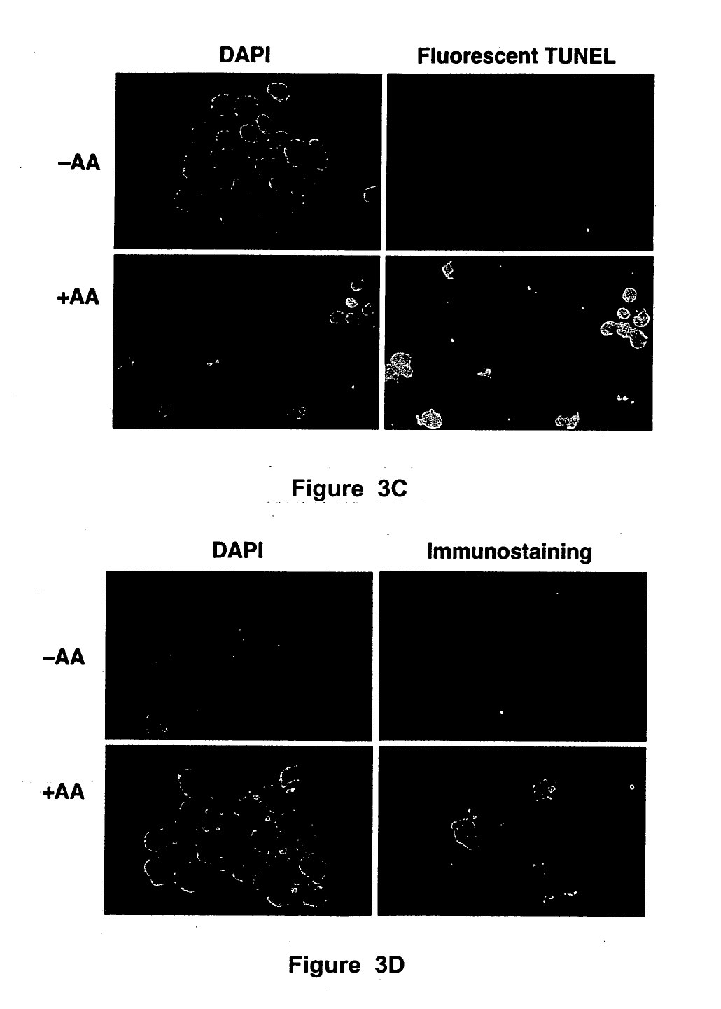

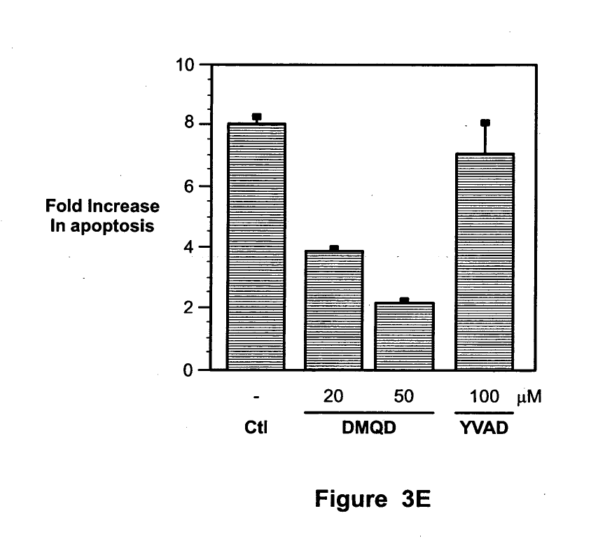

To evaluate the mechanism implicated in the upregulation of AA-utilizing enzymes in tumor tissues, we engineered a cellular model that allowed us to manipulate the cellular free AA level. Initially, we examined the fate of 293 cells exposed to exogenous AA and found that survival fell to 23% of the control as the fatty acid concentration was increased (FIG. 3A). The portion of cell death attributable to apoptosis was estimated in an assay that detects the formation of mono- and oligo-nucleosomes. A high level (18-fold over the control) was detected when the cells were exposed to 300 μM AA, but no increase was observed when they were exposed to palmitic acid (PA) (FIG. 3B). The control cells had intact nuclei, while the cells treated with AA showed nuclear condensation, chromatin fragmentation, and a high level of DNA strand breaks (FIG. 3C). Thus, AA induces apoptosis in a concentration-dependent manner. We postulated that activation of caspase 3 might be involved in the AA-induced apoptosis, since a caspase 3, but not caspase 1 (ICE), mediated pathway is involved in TNFα-treated cells, in which endogenous AA release is stimulated. See S. Bourteele et al., 273 J. Biol. Chem. 31245-31251 (1998). We detected increased caspase 3-like activity in AA-treated cells in a time- and concentration-dependent manner compared to control cells—the maximal increase was ˜4.5 fold and the peak was at 17 hours (not shown). Activation of caspase 3 in individual cells was detected with an antibody specific for activated caspase 3. The staining pattern revealed that the level of activated caspase 3 correlated with the extent of chromatin fragmentation (FIG. 3D). We next added inhibitors of either caspase 1/4 (z-YVAD-fmk) or caspase 3 (z-DMQD-fmk) to the cells before exposing them to exogenous AA. We found that the caspase 3 inhibitor, but not the caspase 1/4 inhibitor, significantly blocked apoptosis (P<0.001 at both concentrations) (FIG. 3E). This indicates that activation of caspase 3 is an essential downstream event in AA-induced apoptosis.

### Example 4

### Activation of AA-Utilizing Pathways Prevents AA-Induced Apoptosis by Reducing the Level of Cellular Free AA

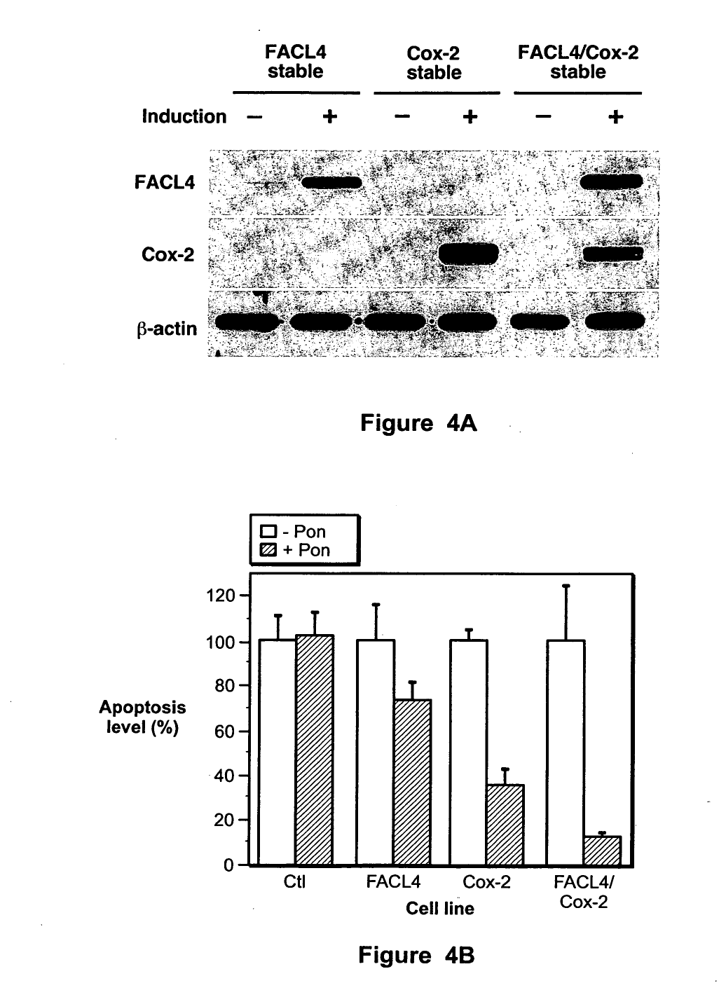

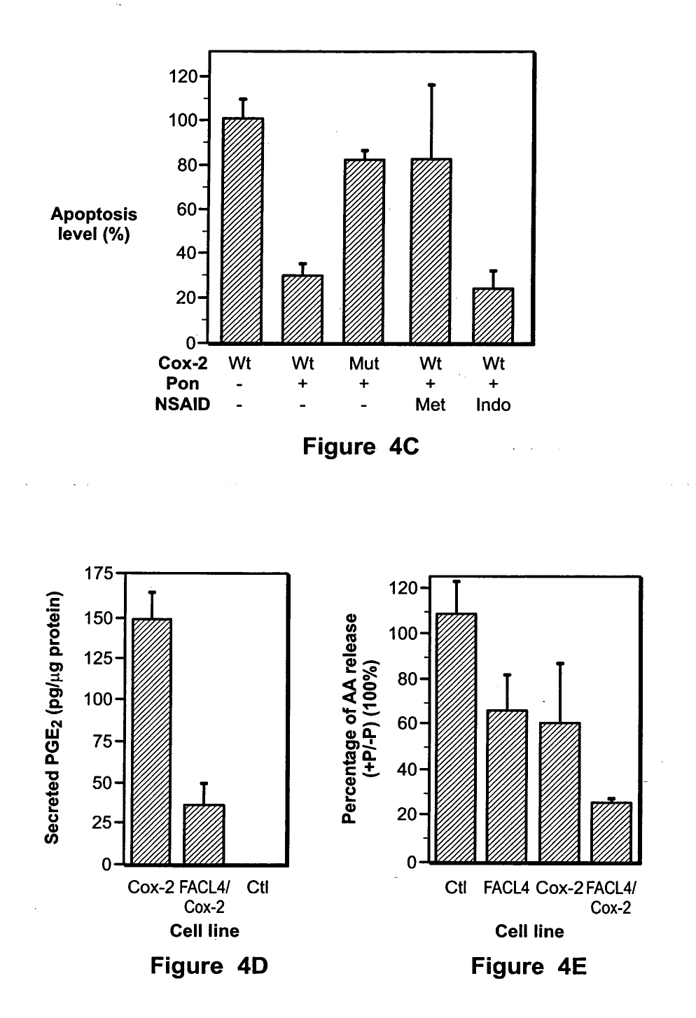

We next constructed stably transfected lines that express human FACL4, human COX-2, or both upon induction. The expression level of FACL4, COX-2, or both upon induction was detected in the stable lines by Western blotting (FIG. 4A). In addition, we isolated lines with inducible expression of a mutant COX-2 (L547K) that lacks catalytic activity (not shown). We then tested if over-expression of FACL4 and/or COX-2 prevented AA-induced apoptosis. A reduced level of apoptosis was observed in cells that over-expressed FACL4 or COX-2 compared to control cells (FIG. 4B). The reduction in apoptosis was not a side effect of ponasterone, the agent used to induce expression of the enzymes, because the control cells harboring the empty vector generated similar levels of signals with or without induction. The attenuation of apoptosis was much more pronounced when cells over-expressed both FACL4 and COX-2 (FIG. 4B). We conclude that over-expression of FACL4 or COX-2 partially prevents AA-induced apoptosis, and simultaneous over-expression of both enzymes has an additive effect.

We next asked if other polyunsaturated fatty acids induced apoptosis and observed that eicosapentaenoic acid (EPA) and docosahexaenoic acid (DHA) were effective. Both of these fatty acids are substrates for COX-2 although DHA is not as good as either AA or EPA. See W. L. Smith et al., 271 J. Biol. Chem. 33157-33160 (1996). The fold-increase in apoptosis was 8.5-, 9.1-, 15.1- and 4.2-fold after treatment with 200 μM AA, 200 μM EPA, 100 μM DHA and 200 μM PA, respectively. By inducing COX-2 over-expression, apoptosis in response to AA was reduced by 36.7% (n=3, P<0.001), to EPA by 34.0% (n=3, P<0.01), to DHA by 18.5% (n=3, P>0.05), and to PA by only 8.1% (n=3, P>0.05). Thus, prevention of apoptosis by over-expression of COX-2 correlated with the suitability of the fatty acid as substrate. To test whether the protective effect of COX-2 required its catalytic activity, as suggested by the results with different fatty acids, we compared the level of apoptosis in cells with wild type COX-2 to that in cells with an inactive mutant COX-2 (L547K). In contrast to wild type COX-2, over-expression of the mutant COX-2 did not inhibit apoptosis (FIG. 4C). We next tested the effect of NSAIDs on this response and found that 6-methoxy-2-naphthylacetic acid (50 μM), a selective inhibitor of COX-2, abolished the prevention of apoptosis by COX-2. In contrast, indomethacin (10 μM, a concentration that inhibits Cox-1 but does not inhibit COX-2) (See E. A. Meade et al., 268 J. Biol. Chem. 6610-6614 (1993)) did not alter the apoptosis response (FIG. 4C). Thus, inhibition of COX-2 had a pro-apoptotic effect.

The protection from apoptosis could have resulted from the extensive removal of free AA, or alternatively, the production of metabolites. To distinguish between these two mechanisms, we performed several experiments: first, we compared the secretion of PGE₂, the major product of the COX-2 pathway, in the COX-2 single and the FACL4/COX-2 double stable lines. The double stable cells secreted ˜28% of the amount of PGE₂ secreted by the COX-2 single stable cells (FIG. 4D). This most likely resulted from competition between COX-2 and FACL4-mediated pathways for the pool of free AA. This result strongly favors the interpretation that depletion of AA, and not the generation of products, was responsible for the prevention, since the double stable cells prevented apoptosis more effectively than the COX-2 single stable cells even though they produced a much lower level of prostaglandins. Furthermore, we directly added PGE₂ (50 μM) and PGJ₂ (5 μM) to the cells and found that they did not improve cell viability in the presence of exogenous AA (not shown). Another possibility was that the prevention resulted from an alteration in HETE synthesis via the LOX pathways because of the decreased availability of free AA in the double stable cells. We detected 15-LOX in 293 cells and found that the LOX inhibitor NDGA induced apoptosis, as reported previously (See D. G. Tang et al., 93 Proc. Natl. Acad. Sci. 5241-5246 (1996)), moreover, addition of 15-HETE did not affect cell survival in 293 cells (not shown). Taken together, these results support the hypothesis that a high level of free AA triggers apoptosis, while its depletion blocks apoptosis. To obtain direct evidence, we next estimated the relative amounts of free AA in the different cell lines by measuring the amount released into the medium. See S. Jayadev et al., 269 J. Biol. Chem. 5757-5763 (1994). After induction of the “sink”, AA was reduced to 67%, 61% and 26% in the FACL4, COX-2 and the double stable cells, respectively, compared to the counterparts without induction (FIG. 4E).

### Example 5

### The Arachidonic Acid Metabolic “Sink” Also Prevents TNFα-Mediated Killing

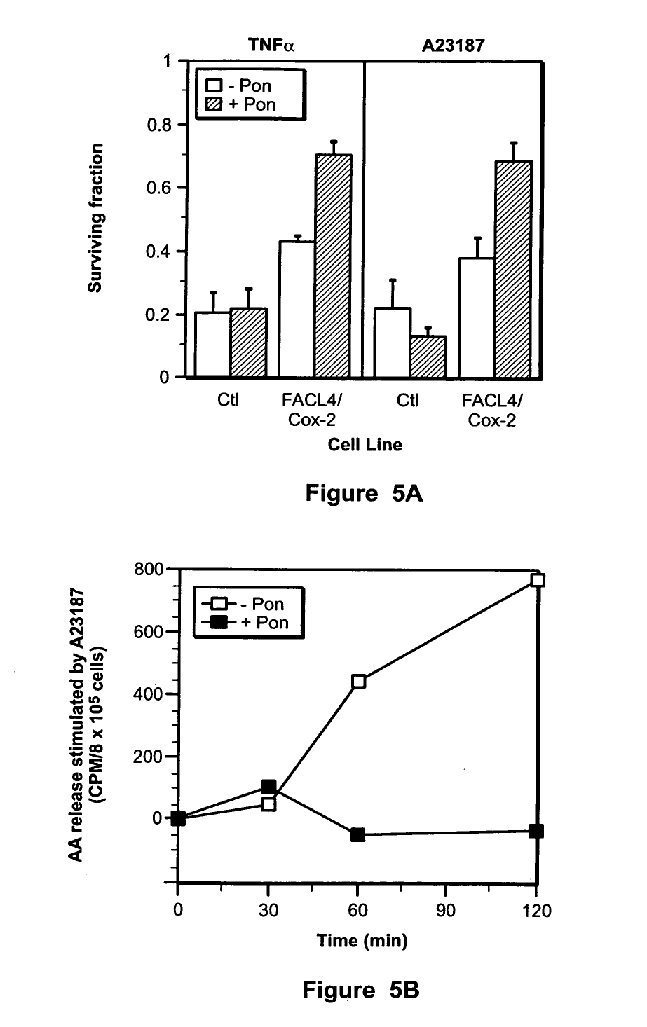

We next tested if the “sink” for AA could apply to prevent cell death in other circumstances, for example, TNFα-mediated killing. TNFα exerts cytotoxic and cytostatic effects against certain tumor cells, and release of AA by cPLA₂ clearly has been implicated in this process. See M. Hayakawa et al., 268 J. Biol. Chem. 11290-11295 (1993); and S. Jayadev et al., 272 J. Biol. Chem. 17196-17203 (1997). In the cells that overexpressed FACL4 or COX-2, the cell survival after TNFα treatment was increased moderately (not shown), and it was significantly improved in the FACL4/COX-2 stable cells: the surviving fraction increased from 43.5% to 70.5% (n=3, P<0.001) (FIG. 5A). We observed that cell survival in the double stable cells was better than that in control cells even without induction. This probably resulted from a slight leak in promoter regulation of the cDNA constructs. We also examined cell survival in response to another agonist, Ca²⁺ ionophore A23187, which stimulates AA release by cPLA₂ (See L.-L. Lin et al., 89 Proc. Natl. Acad. Sci. 6147-6151 (1992)): survival was higher in induced FACL4 or COX-2 stable cells than in their uninduced counterparts (not shown). The improvement was even more pronounced in the double stable cells: cell survival rose from 37.8% to 67.8% (t test, n=3, P<0.01) (FIG. 5A). We repeatedly observed synergistic actions by over-expression of both FACL4 and COX-2. We also found that the COX-2 specific inhibitor (6-methoxy-2-naphthylacetic acid) partially abolished the improvement in cell survival in the double stable cells (not shown). To further test whether the improvement of cell survival was a consequence of reduction of the level of free AA, we performed a pulse-chase experiment and measured the AA released to the medium after A23187 treatment. Control cells released a large amount of AA in response to A23187, but its release was completely abolished by over-expression of FACL4 and COX-2 (FIG. 5B). This demonstrated that diversion of AA into metabolic pathways (a sink) resulted in a low cellular concentration even under conditions of a strong stimulus for AA release. These results support that a rise in the level of AA is a key step in TNFα- and A23187-mediated killing and that free AA mediates cell death signaling initiated by a variety of stimuli.

### Example 6

### The AA-Induced Apoptosis Is Suppressed by Antioxidants and Bcl-2

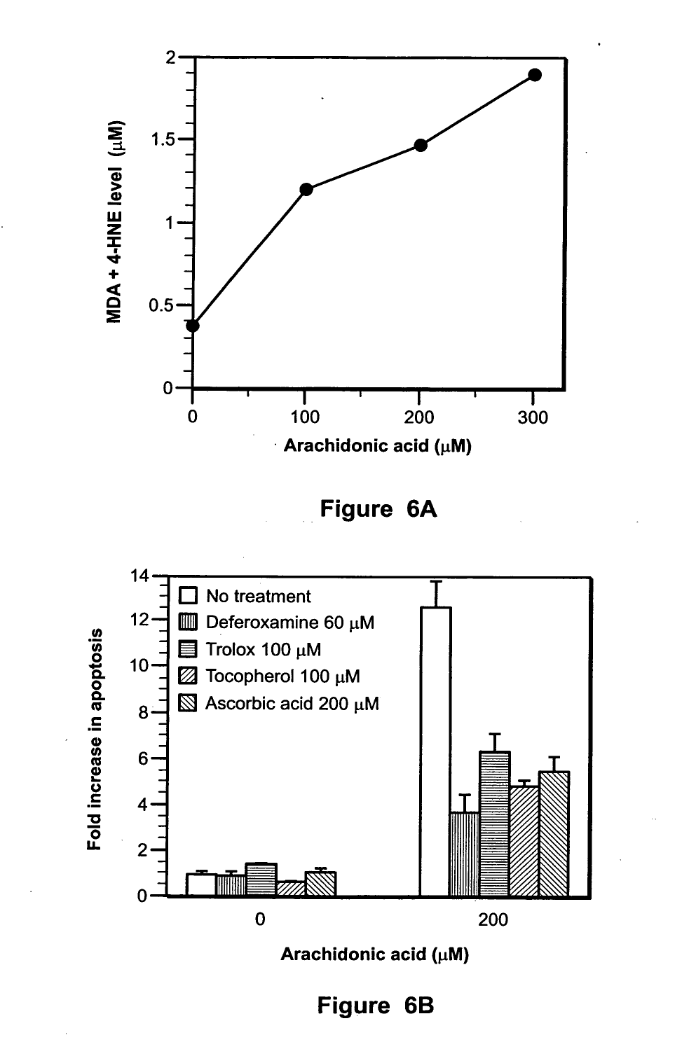

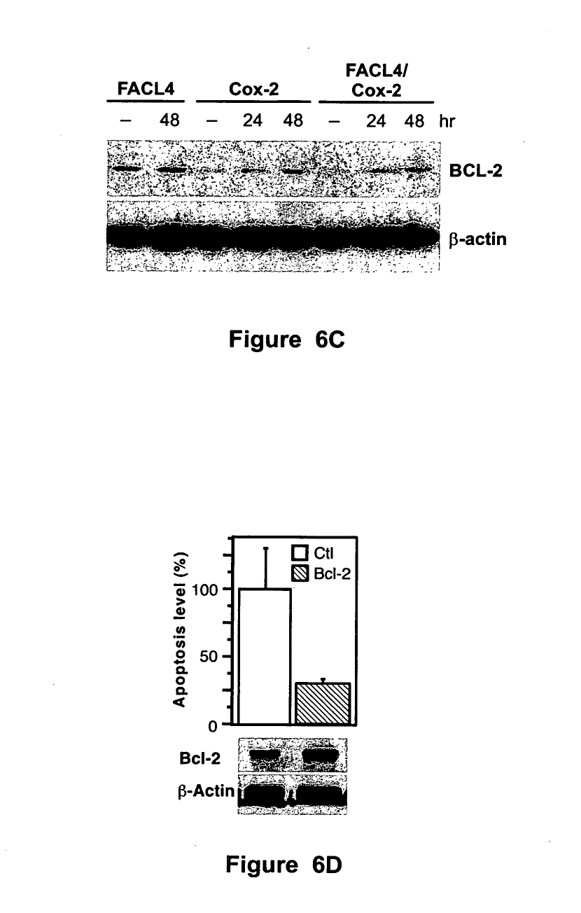

Lipid peroxidation often is a component of apoptosis (See D. M. Hockenbery et al., 75 Cell 241-251 (1993)), and we observed enhanced formation of malondialdehyde and 4-hydroxy-2(E)-nonenal in cells exposed to AA (FIG. 6A). Moreover, apoptosis decreased significantly when the cells were pretreated with antioxidants including deferoxamine, ascorbic acid, trolox and tocopherol (FIG. 6B). We also tested apo-transferrin, a membrane-impermeable antioxidant, and found that it did not alter AA-induced apoptosis (not shown). Thus, antioxidants partially protect against apoptosis by blocking an intracellular oxidative process. Our results suggest that reactive oxygen species are involved in AA-induced apoptosis, which might include peroxidation of the AA, perhaps via non-enzymatic mechanisms. The caspase 3 inhibitor did not block the generation of peroxidized lipids, which places the accumulation of peroxidized lipids upstream of caspase 3 activation (not shown).

Bcl-2 is an anti-apoptotic protein and one of its effects is to suppress lipid peroxidation. See D. M. Hockenbery et al., 75 Cell 241-251 (1993). We transfected cells with a Bcl-2 cDNA and found that it reduced the AA-induced apoptosis and that the reduction correlated with the expression level of Bcl-2 (FIG. 6C). This raised the possibility that the protection from apoptosis observed in the cells with an AA-sink might have resulted from increased expression of Bcl-2 in those cells. Indeed, we found that the expression levels of Bcl-2 increased moderately when FACL4 and/or COX-2 are over-expressed (FIG. 6D). Thus, the apoptosis pathway initiated by AA involves lipid peroxidation and can be suppressed by Bcl-2, but the protective effect provided by expression of AA-metabolizing enzymes lies upstream of these steps—by regulating the amount of free AA available.

1. A method for evaluating the presence or absence of colon cancer cells in a sample of a suspect tissue comprising the steps of:
a. isolating the sample of the suspect tissue;
b. evaluating the level of FACL4 expression in the cells of the tissue; and
c. comparing the level of FACL4 expression in the cells of the tissue with those of a control cell.

2. The method of claim 1, wherein the method of evaluating levels of FACL4 expression in the cells of the tissue is quantitative RT-PCR.

3. The method of claim 1, wherein the method of evaluating levels of FACL4 expression in the cells of the tissue is immunostaining or in situ hybridization.

4. The method of claim 1, wherein the method of evaluating levels of FACL4 expression in the cells of the tissue is Western blotting.

5. The method of claim 1, wherein the control cell is from a non-cancerous intestinal cell line.

6. The method of claim 1, wherein the control is the intestinal epithelial line Int 407.

7. A method for diagnosing colon cancer comprising the steps of:
a. isolating a sample from a region of suspected cancerous tissue;
b. isolating a sample from a region of similar tissue thought to be non-cancerous;
c. evaluating levels of FACL4 in each of the samples; and
d. comparing the levels of FACL4 in the suspected tissue with those in the non-suspect tissue.

8. The method of claim 7, wherein the method of evaluating levels of FACL4 expression in the cells of the tissue is quantitative RT-PCR.

9. The method of claim 7, wherein the method of evaluating levels of FACL4 expression in the cells of the tissue is immunostaining.

10. The method of claim 7, wherein the method of evaluating levels of FACL4 expression in the cells of the tissue is immunofluorescence.

11. A method of screening for potential cancer chemotherapeutic agents comprising the steps of:
a. administering a test compound to an animal; and
b. measuring inhibition of the function of the FACL4 gene product in the animal, wherein a test compound which inhibits the function of the FACL4 gene product is a potential cancer chemotherapeutic agent.

12. The method of claim 11, wherein the animal is a mouse.

13. The method of claim 11, wherein the animal is a mouse in which one or both FACL4 alleles have been mutated.

14. The method of claim 11, wherein the animal is a transgeneic mouse that overexpresses the FACL4 gene product in one or more tissues.

15. The method of claim 11, wherein the animal is a mouse bearing a xenografted tumor.

16. A method for inhibiting FACL4 activity in a human, comprising administering a compound which inhibits FACL4 to a human in need of such treatment.

17. The method of claim 16 wherein the compound is triacsin C.

18. A method for inhibiting FACL4 activity in a human, comprising administering a compound which inhibits FACL4 to a human in need of such treatment, wherein the activity of the compound does not result in significant toxic side effects to the human.

19. A method for inhibiting FACL4 activity in a human, comprising administering a compound that inhibits FACL4 to a human in need of such treatment, wherein the ability of the compound to inhibit FACL4 is determined by:
a. contacting a cell with a test compound, wherein the cell expresses or overexpresses a FACL4 gene product; and
b. measuring inhibition of the function of the FACL4 gene product in the cell.

20. A method for treating colon cancer in a human suffering therefrom, comprising administering a compound that inhibits FACL4 activity to a human in need of such treatment.

21. A method for treating colon cancer in a human suffering therefrom, comprising:
a. administering a compound which inhibits COX-2; and
b. administering a compound which inhibits FACL4.
